(19) United States
(12) Patent Application Publication
(10) Pub. No.: US 2025/0161078 A1
(43) Pub. Date: May 22, 2025

(54) Prosthetic Ankle Joint

(71) Applicant: Alps South, LLC, St. Petersburg, FL (US)

(72) Inventors: Aldo Laghi, Pinellas Park, FL (US); David Carson, Clearwater, FL (US); Jakob W. McClelland, Gulfport, FL (US); Nathaniel Vint, Palm Harbor, FL (US)

(21) Appl. No.: 18/805,124

(22) Filed: Aug. 14, 2024

Related U.S. Application Data
(63) Continuation of application No. 18/802,802, filed on Aug. 13, 2024.
(60) Provisional application No. 63/568,589, filed on Mar. 22, 2024.
(60) Provisional application No. 63/601,784, filed on Nov. 22, 2023.

(51) Int. Cl. A61F 2/66; A61F 2/50

(52) U.S. Cl. CPC A61F 2/6607; A61F 2002/5072; A61F 2002/6614

(57) ABSTRACT

A prosthetic ankle joint used with a prosthetic foot to allow for a non-linear spring to be used for energy absorption and return capacity. Specifically, the ankle joint comprises a main body that connects to the shank of a prosthetic foot using mounting bolts that further connects to a pyramid adaptor using a non-linear spring. The use of a non-linear spring as an attachment member allows for movement of the attachment point in relation to the foot while retaining the added benefits of a conical spring and the nonlinear compression it has. The non-linear spring of the present invention is preferably a progressive rate spring and is preferably conical but may other shapes that increase stiffness with deflection. The spring rate is determined by the weight of the patient and their activity level.

### CROSS-REFERENCE TO RELATED APPLICATIONS

This application is a continuation of U.S. patent application Ser. No. 18/802,802 titled “Prosthetic Ankle Joint,” filed Aug. 13, 2024, which claims priority to U.S. Provisional Patent Application No. 63/601,784 titled “Prosthetic Ankle Joint,” filed Nov. 22, 2023, and to U.S. Provisional Patent Application No. 63/568,589 titled “Prosthetic Ankle Joint,” filed Mar. 22, 2024, the disclosures of which are hereby incorporated by reference herein in their entirety.

### FIELD OF THE INVENTION

This invention relates to a prosthetic ankle joint. Specifically, the described invention relates to a prosthetic ankle joint using a non-linear spring and at least one guide pin.

### DESCRIPTION OF BACKGROUND ART

Most prosthetic feet are sold as singular units, designed specifically for the geometry of the foot in question. There are prosthetic feet on the market that utilize different means of vertical loading through urethane, carbon, air pressure, or linear springs to act as shock absorbing and energy return bodies. These bodies either are plagued with maintenance issues and/or have very linear spring rates which effects the gait of the amputee. For example, hydraulic ankles generally feature a failure of seals over time. Furthermore, a linear spring rate results in a very abrupt shock on heel strike with minimal cushioning effect or a loss of energy return from a lower spring rate that may bottom out under high loads like walking quicker or jogging.

Linear springs follow Hooke's Law with regard to the force needed to displace it a certain distance. Nonlinear springs do not follow Hooke's Law and have a non-linear relationship regarding the force needed to displace it a certain distance. Use of a non-linear spring also allows the spring to become a base member for the shock absorption as well as articulation of the ankle joint. Since the conical spring design has a low profile and allows for the individual coils to be stacked or pass by each other in various places, this attribute can be exploited by attaching each end of the spring to the device allowing for the spring to become a moveable member. This moving member will not only have compression for shock absorption but can have rotational forces and angular forces since there can be no guide rod preventing the articulation. The spring design itself can become the limiting factor in not only compression but rotation and articulation as the corresponding spring turns touch each other under different forces.

Therefore, it is an object of this invention to provide an improvement which overcomes the aforementioned inadequacies of the prior art devices and provides an improvement which is a significant contribution to the advancement of the prosthetic ankle joint art.

Another object of this invention is to provide a prosthetic ankle joint that allows for a variety of movement through a variety of activities.

Another object of this invention is to provide for a prosthetic foot unit that does not have a vertical loading component to be fitted with the present invention to allow for a loading pylon attachment to be added.

Another object of this invention is to provide a prosthetic ankle joint that takes advantage of the spring properties of a non-linear spring as compared to a cylindrical spring.

Another object of this invention is to reduce the amount of maintenance necessary that normally comes with the use of linear springs, hydraulics, or pneumatics.

Another object of this invention is to reduce the size of the stroke needed to achieve the same spring return which in turn greatly improves the gait of the patient by keeping their biomechanics in line with how an organic foot/ankle complex would act.

Another object of this invention is to provide a prosthetic that creates a gentle ground reaction at slow ambulation and a stiffer ground reaction when running or jumping.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The long-standing but heretofore unfulfilled need for an apparatus that overcomes the limitations of the prior art is now met by a new, useful, and non-obvious invention. The invention meets the need for a new prosthetic locking attachment system that overcomes the issues inherent in the prior art.

The present invention relates generally to a prosthetic ankle joint used with a prosthetic foot to allow for a non-linear spring to be used for energy absorption and return capacity. Specifically, the ankle joint comprises a main body that connects to the shank of a prosthetic foot using mounting bolts that further connects to a pyramid adaptor using guide pins and a non-linear spring. The use of a non-linear spring as an attachment member can also allow for a multiaxial movement of the attachment point in relation to the foot while retaining the added benefits of a conical spring and the nonlinear compression it has. The non-linear spring of the present invention is preferably a progressive rate spring and is preferably conical but may other shapes that increase stiffness with deflection. The spring rate is determined by the weight of the patient and their activity level. Complementary teeth are alternatively available to prevent rotation.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a top left front perspective view of the present invention;

FIG. 2 is a bottom right rear perspective view of the present invention;

FIG. 3 is a top right rear perspective view of the present invention;

FIG. 4 is a bottom left front perspective view of the present invention;

FIG. 5 is a cross-sectional view of the present invention along line 5-5 of FIG. 1;

FIG. 6 is a cross-sectional view of the present invention along line 6-6 of FIG. 1;

FIG. 7 is an exploded view of the present invention;

FIG. 8 is a perspective view of an alternative embodiment of the present invention;

FIG. 9 is a cross-sectional view of the alternative embodiment shown in FIG. 8 along line H-H;

FIG. 10 is a cross-sectional view of an alternative embodiment of the present invention;

FIG. 11a is a cross-sectional view of a conical spring having a rectangular shape as used in any of the embodiments of the present invention;

FIG. 11b is a cross-sectional view of a conical spring having a triangular shape as used in any of the embodiments of the present invention;

FIG. 11c is a cross-sectional view of a conical spring having a plurality of shapes as used in any of the embodiments of the present invention;

FIG. 12 is a perspective view of an alternative embodiment of the present invention;

FIG. 13 is a cross-sectional view of an alternative embodiment of the present invention;

FIG. 14 is a cross-sectional view of an alternative embodiment of the present invention; and

FIG. 15 is an exploded view of the alternative embodiment shown in FIGS. 12-14.

Similar reference numerals refer to similar parts through the several views of the drawings.

### DETAILED DESCRIPTION OF THE DRAWINGS

The following description is of the best mode presently contemplated for carrying out the invention. This description is not to be taken in a limiting sense but is made merely for the purpose of describing one or more preferred embodiments of the invention. The scope of the invention should be determined with reference to the claims.

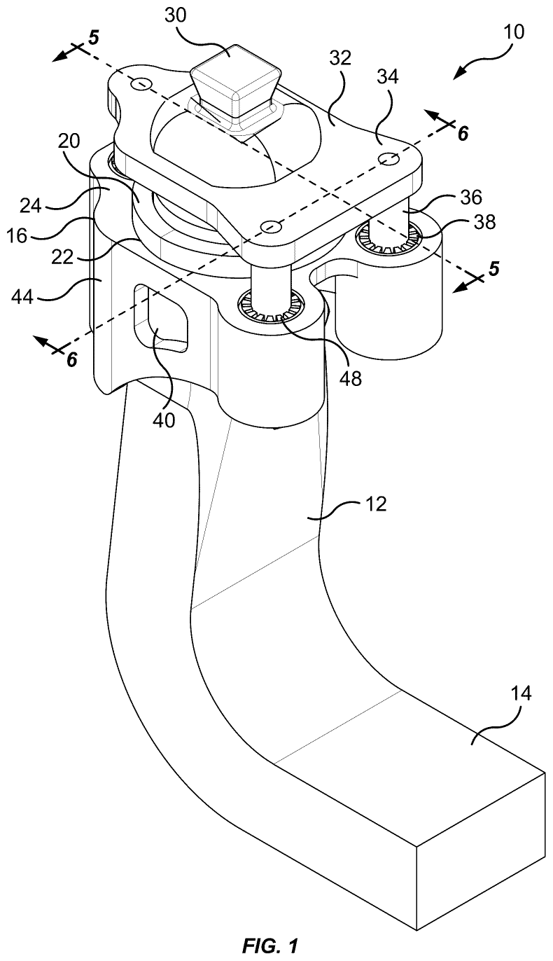

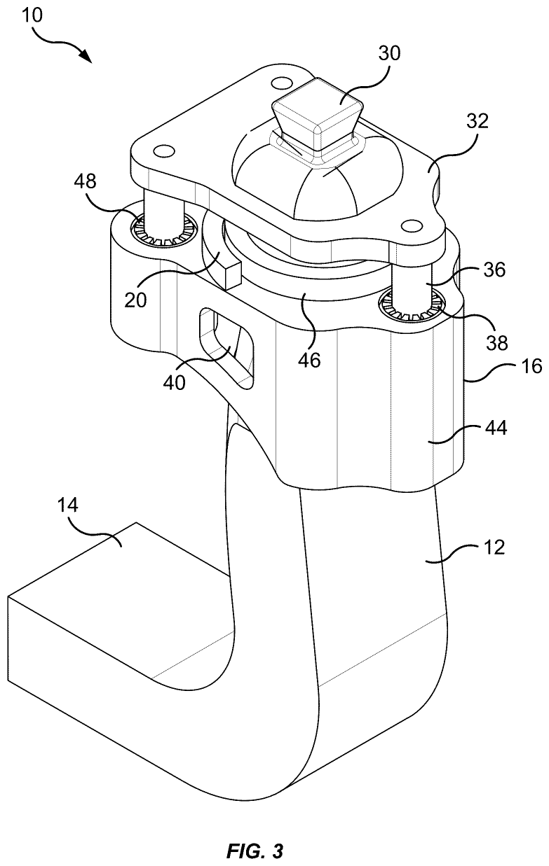

As seen in FIGS. 1 through 4, the ankle joint 10 is attached to the shank 12 of a prosthetic foot 14. The prosthetic foot 14 shown here is shown as a generic shape to account for the fact that prosthetic feet come in a variety of shapes and sizes and the ankle joint 10 of the present invention is designed to fit onto a multitude of types of prosthetic feet. The prosthetic foot 14 may be carbon reinforced, glass reinforced, or otherwise reinforced to account for a patient's increased activity.

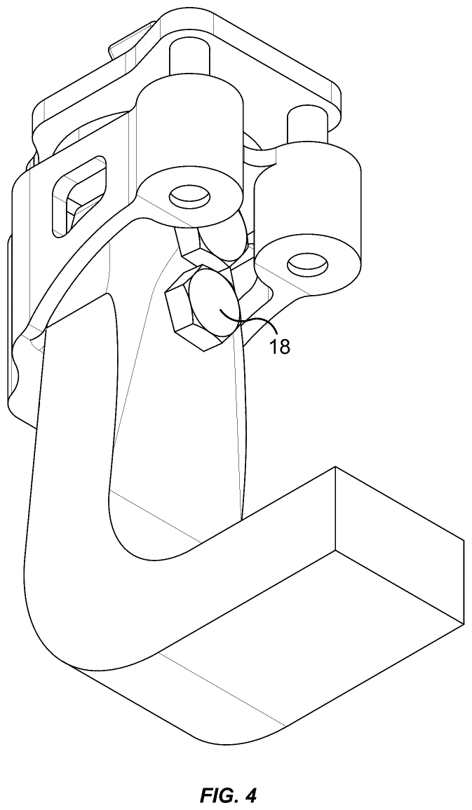

The ankle joint 10 has a main body 16 that is preferably machined according to the prosthetic foot 14 it is being attached to. The main body 16 attaches to the prosthetic foot using a plurality of mounting bolts 18 (as shown in FIG. 4). The main body 16 has a centrally located non-linear spring 20. The non-linear spring 20 preferably folds within itself to form a flat circle when fully compressed. The spring base 22 and the spring top 26 is preferably compressed between the upper body face 24 of the main body 16 and the bottom pyramid adaptor face 28 of the pyramid adaptor plate 32 thereby holding the non-linear spring 20 in place. This allows for adjustable pre-compression of the non-linear spring 20 which is beneficial to the patient because the non-linear spring 20 can be adjusted based on activity. Alternatively, the non-linear spring 20 can be epoxied, welded, or otherwise physically attached to the main body 16 and the pyramid adaptor plate 32. A pyramid adaptor 30 is attached to the top pyramid adaptor face 34 of the pyramid adaptor plate 32. The pyramid adaptor 30 allows for the ankle joint 10 to interface with a leg prosthesis.

The pyramid adaptor plate 32 has at least one guide pin 36 and, in this embodiment, three guide pins 36 that connect it to the main body 16 of ankle joint 10. These guide pins 36 feed into guide pin holes 38 in the main body 16 which each have a plurality of bearings 48 within. These bearings 48 allow for the guide pins 36 to travel vertically in the guide pin holes 38 when downward pressure is created. Proximal end 50 of guide pins 36 are attached to bottom pyramid adaptor face 28 of the pyramid adaptor plate 32 and float within the guide pin holes 38 while in use. Particularly in the situation as shown in FIG. 1 wherein there are three guide pins 36 and three guide pin holes 38, downward pressure, such as by walking or running, will displace the guide pin an appropriate amount for the patient to maintain balance and the non-linear spring 20 will have increased resistance when a greater downward force is applied because of the preferred progressive rate.

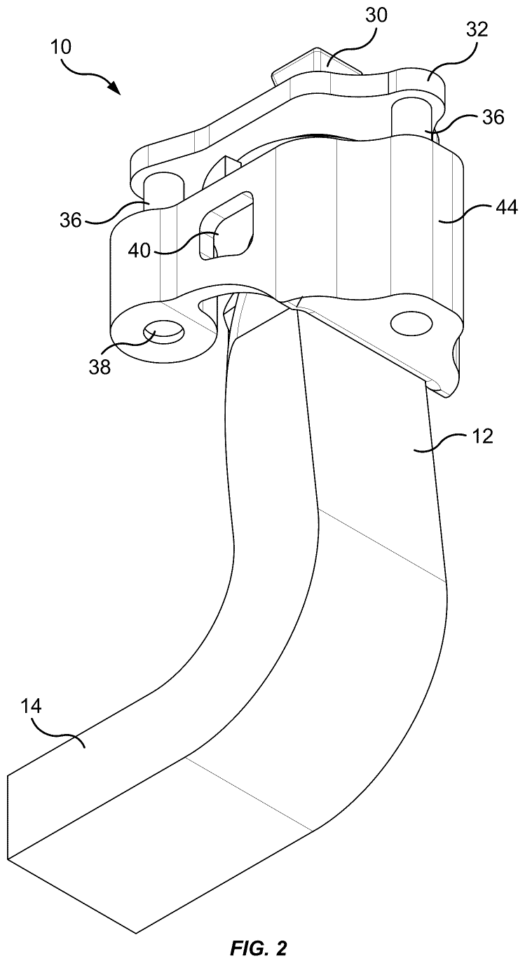

As shown in FIG. 2, the main body 16 may have a plurality of voids 40 throughout so as to lower the overall weight of the ankle joint 10.

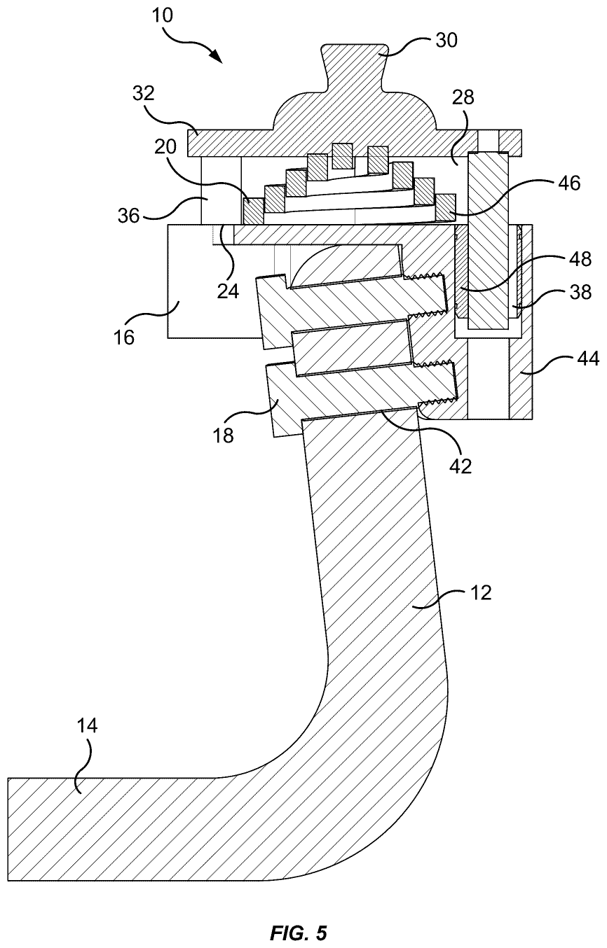

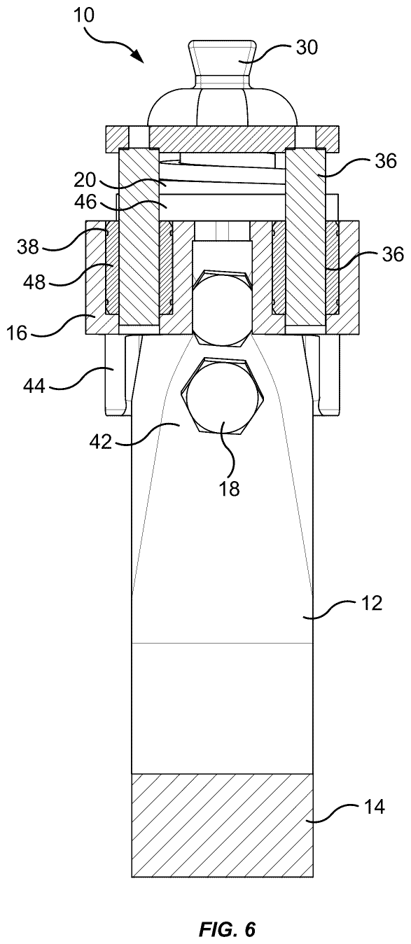

FIGS. 5 and 6 provide a better understanding of how the ankle joint 10 of the present invention is oriented. The mounting bolts 18 are fed through the shank 12 and mate with interior voids 42 of the main body 16. Similarly, the guide pin 36 and guide pin hole 38 are shown penetrating the back body 44 of the main body 16. The back body 44 extends further down the shank 12 so that the mounting bolts 18 have something to which they can mount.

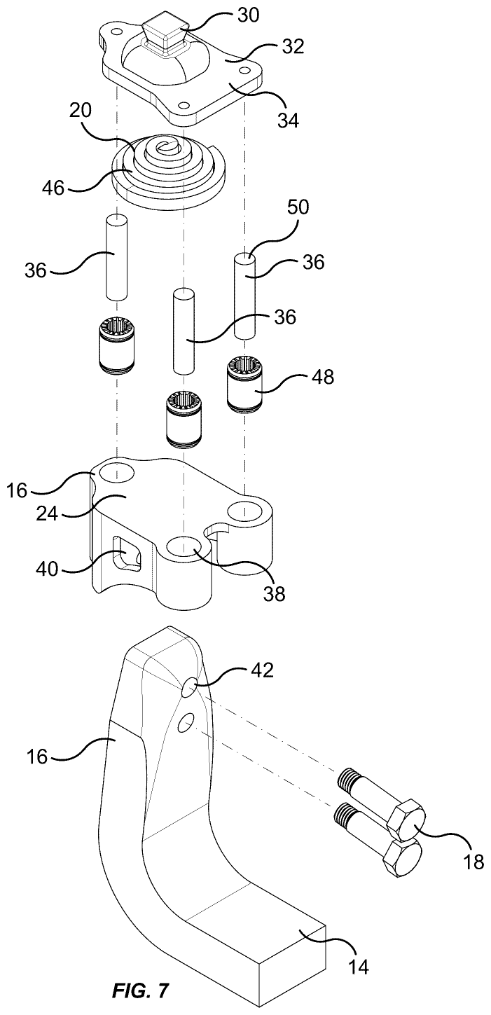

As seen in the exploded view of FIG. 7, the non-linear spring 20 is preferably conical in shape but may be any shape that creates a progressive rate of spring stiffness with increasing deflection. The non-linear spring 20 preferably has a structure that allows for the coils 46 to fit within one another so as to decrease the total height needed for the ankle joint 10. The non-linear spring 20 can have uniform or varied cross sections to change certain attributes of the spring. A conical spring is nonlinear by nature but varying the cross section in thickness or shape can further exploit these benefits.

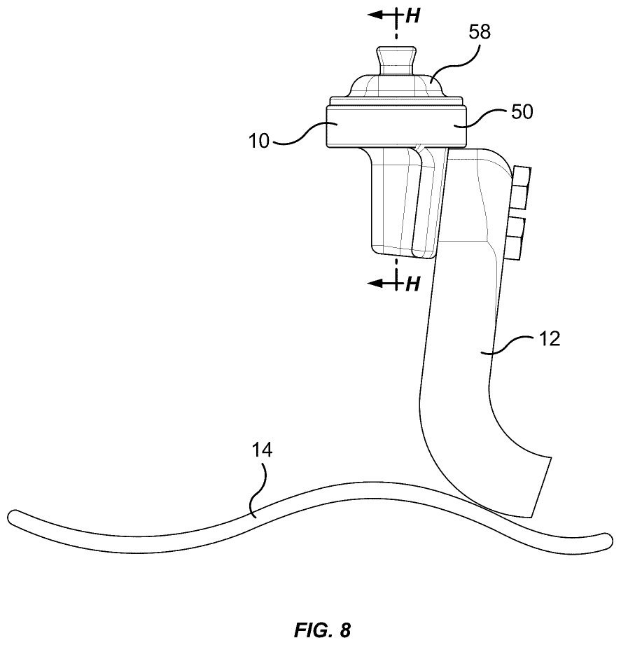

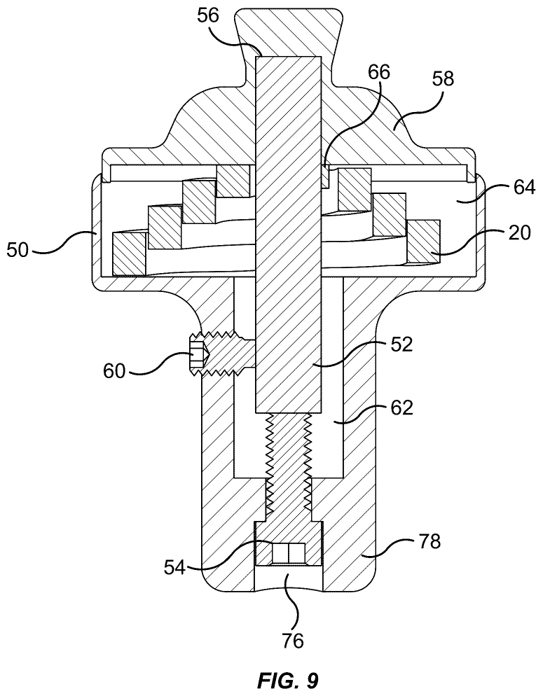

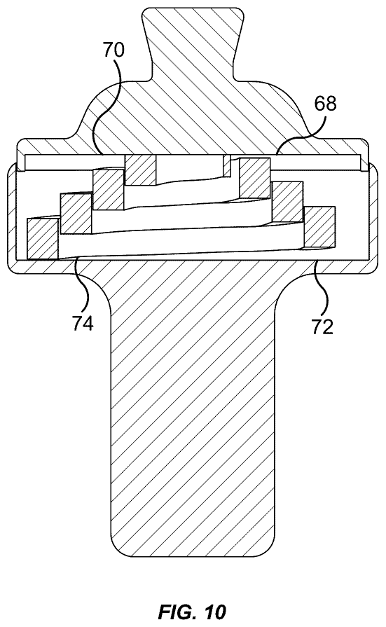

An alternative embodiment is shown in FIG. 8 and FIG. 9. In this embodiment, a main body 50 having a main body housing 90 wherein the main body housing 90 contains the non-linear spring 20 in a body cavity 64. A single, centrally located internal guide rod 52 is held in place using a tension screw 54 that pushes the internal guide rod 52 into a void 56 of a pyramid adaptor 58 through a spring aperture 66 thereby compressing the non-linear spring 20. The tension screw 54 enters the main body 50 through an aperture 76 at the main body distal end 78 and is stopped by a collar 88. The aperture 76 continues until the main body platform 84. The internal guide rod 52 is held in place and rotation/lateral movement is limited through the use a set screw 60 and internal bushing 62. Alternatively, as shown in FIG. 10, no guide rod is necessary and the non-linear spring 20 may be fastened to a bottom pyramid adaptor face 68 at a proximal end 70 of the non-linear spring 20 and fastened to an internal body face 72 at a distal end 74 of the non-linear spring 20. This type of connection still allows for compression and prevents rotation by virtue of the non-linear spring 20 being connected to the pyramid adaptor 58 and main body platform 84.

The anti-rotation of the present invention can provide stability in some patients to prevent the rotational moment induced when certain movements are applied. Rotation can be further prevented by the inclusion of a slot within the shaft that is controlled by a pin in the housing. Other methods are non-circular shafts that inherently cannot rotate within a corresponding bushing. A limiting rotation can be a groove within the shaft that is controlled by a detente that will limit the ability to rotate until a minimum force is met and overcome to allow the rotation. This can then be user adjusted to increase or decrease the minimum rotational force for patient comfort and compliance.

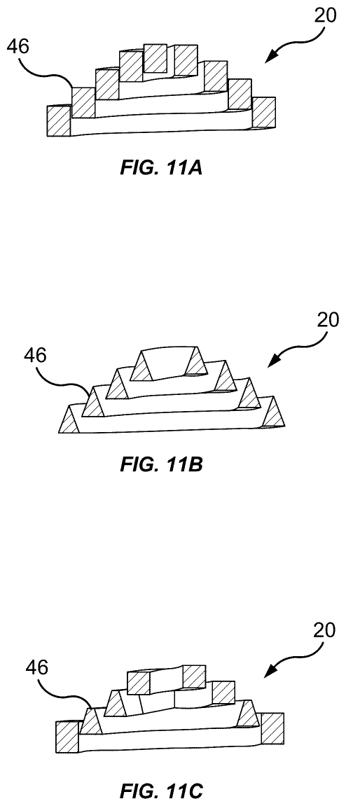

As seen in FIGS. 11a-11c, the non-linear spring 20 can have coils 46 that are a variety of shapes. In these related embodiments, the non-linear spring 20 may have a uniform cross-section but have a rectangular, square, circular, triangular, trapezoidal or oval shape which allows for different spring rates due to packaging size. For example, as seen in FIG. 11a and FIG. 11b respectively, the coils 46 may be rectangular or triangular in shape. The stiffness of the spring can be redefined simply by changing its shape. The overall compression of the spring is effected by shape as well since a conical spring is designed to have the concentric spring coils pass through each other. Depending on the spring cross-section, a limited effect can be controlled through physical contact or due to the cross sectional shape itself. In a more advanced conical spring, a varied cross section can change the attributes outside of providing attachment points. For example, as seen in FIG. 11c, larger coils 46 may be of a rectangular shape while the mid-section can be triangular to allow for a limited or expansive articulation while the upper most coils can be a square to provide the most resistance at the end of spring travel.

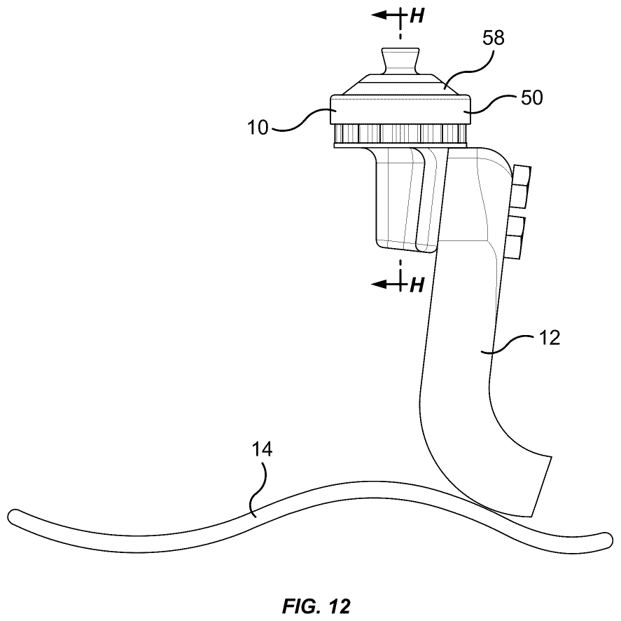

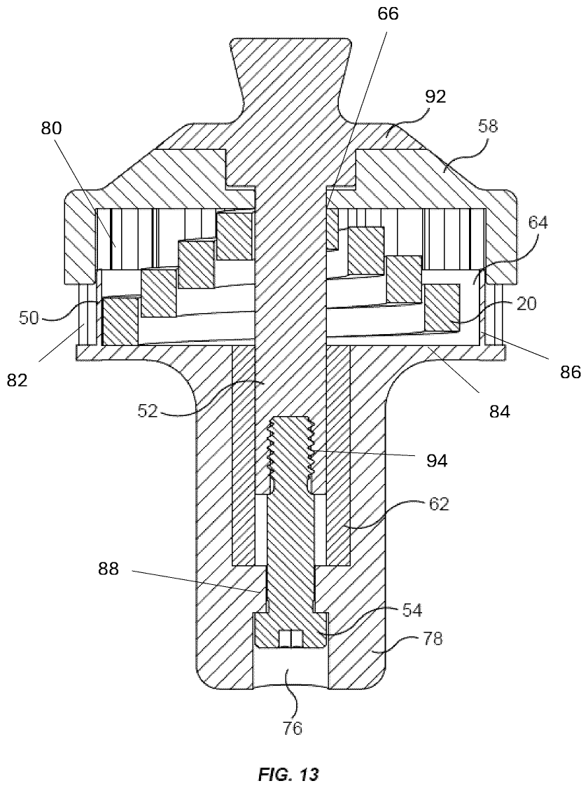

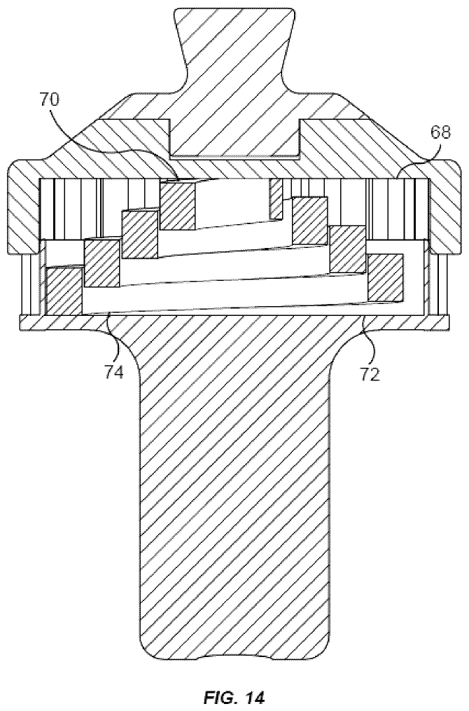

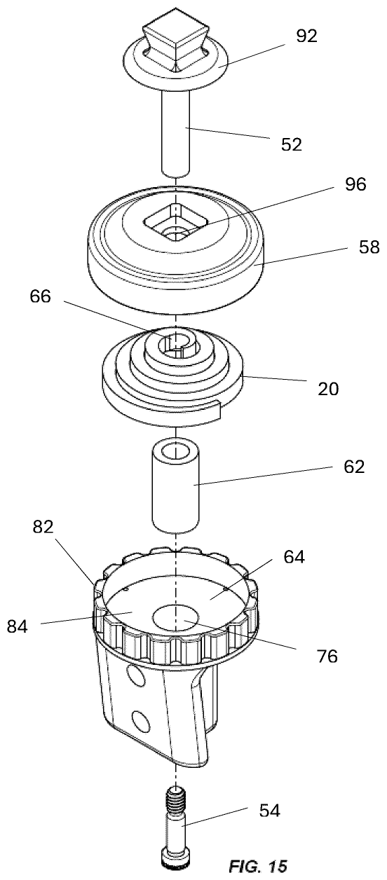

An alternative embodiment is provided in FIGS. 12-15. In this embodiment, the ankle joint 10 is further limited in rotation through the use of a plurality of adaptor teeth 80 around the interior circumference 86 of the pyramid adaptor 58. The adaptor teeth 80 have complementary main body teeth 82 circumferentially located around the main body platform 84 that interlock when the pyramid adaptor 58 is joined with main body 50 and tightened into place using tension screw 54. This embodiment may also use a set screw 60 to further limit rotation as needed. Additionally, the pyramid adaptor 58 may have a separate pyramid head 92 that forms a unitary piece with the internal guide rod 52 such that the tension screw 54 engages with a screw aperture 94 pulling the pyramid head 92, which has a pyramid aperture 96, and internal guide rod 52 into compression with the main body 50.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A prosthetic ankle joint comprising:
a main body having a main body distal end and a main body housing, wherein the main body housing further comprises a body cavity housing a non-linear spring having a spring distal end and a spring proximal end, a main body platform;
a pyramid adaptor comprising a bottom pyramid adaptor face; and
wherein the non-linear spring is attached to the main body at the spring distal end and the pyramid adaptor at the spring proximal end.

2. The prosthetic ankle joint of claim 1 wherein the non-linear spring has a cross-sectional shape of rectangular, square, circular, triangular, trapezoidal or oval shape.

3. A prosthetic ankle joint system comprising:

4. A prosthetic foot having a shank; and

5. An ankle joint attached to the shank, wherein the ankle joint further comprises a main body having a main body distal end and a main body housing, wherein the main body housing further comprises a body cavity housing a non-linear spring having a spring distal end and a spring proximal end, a main body platform, a pyramid adaptor comprising a bottom pyramid adaptor face, and wherein the non-linear spring is attached to the main body at the spring distal end and the pyramid adaptor at the spring proximal end.

6. The prosthetic ankle joint of claim 3 wherein the non-linear spring has a cross-sectional shape of rectangular, square, circular, triangular, trapezoidal or oval shape.
